(19) United States
(12) Patent Application Publication
(10) Pub. No.: US 2007/0140994 A1
(43) Pub. Date: Jun. 21, 2007

(54) Substituted hydrocarbyl functional siloxanes-silicone resin

(75) Inventors: Cassandre Michelle Fecht, Midland, MI (US); Heide Marie Vandort, Sanford, MI (US); Lori Ann Stark-Kasley, Midland, MI (US); Adriana Urrutia, Mexico City (MX)

Correspondence Address: DOW CORNING CORPORATION CO1232, 2200 W. SALZBURG ROAD, P.O. BOX 994, MIDLAND, MI, 48686-0994, US

(73) Assignee: DOW CORNING CORPORATION, Midland, MI (US)

(21) Appl. No.: 10/592,714

(22) PCT Filed: Mar. 30, 2005
(86) PCT No.: PCT/US05/10860

§ 371 Date: Sep. 13, 2006

Related U.S. Application Data
(60) Provisional application No. 60/563,662, filed on Apr. 20, 2004.

(51) Int. Cl. A61K 8/89

(52) U.S. Cl. 424/59; 424/70.12; 424/64

(57) ABSTRACT

Sunscreen compositions containing a hydrocarbyl functional organopolysiloxane are disclosed. The hydrocarbyl functional organopolysiloxane contains at least one siloxy unit of the formula —R²OCH₂CH₂OH, where R² is a divalent hydrocarbon group containing 2 to 6 carbon atoms. The inclusion of the hydrocarbyl functional organopolysiloxane in the sunscreen composition improves the sun-protection factor (SPF) of the sunscreen component and various personal, medical and household care compositions containing the sunscreen composition.

### FIELD OF THE INVENTION

The present invention relates to compositions containing a hydrocarbyl functional organopolysiloxane and an organopolysiloxane resin. The hydrocarbyl functional organopolysiloxane contains at least one siloxy unit of the formula —R²OCH₂CH₂OH, where R² is a divalent hydrocarbon group containing 2 to 6 carbon atoms. The combination of the hydrocarbyl functional organopolysiloxane and organopolysiloxane resin provides a base composition with improved film properties that can be used in various personal, medical, automotive and household care compositions.

### BACKGROUND OF THE INVENTION

A successful color cosmetic composition usually encompasses 3 key elements to optimize performance of the final product. These elements can be described as; the color film formed on the skin, the pigments that are used to formulate the product, and the structure or texture of the color cosmetic that has been selected. Each of these elements require a specific attention about its fundamental behavior, as each interacts with each other causing a different behaviors and results from what the formulator expects, some times causing defects or poor performance in the final color product.

Perhaps one of the most critical requirements for a color cosmetic is the film formation. If the film is not uniform, the optimum color development of the final product will not occur, the film gloss will be affected and the final color cosmetic product will show poor performance.

Films can be formed in several ways. The simplest method is to dissolve all the ingredients in a solvent or solvents to achieve the desired application viscosity, then apply the product on the skin and then allow the solvent to evaporate. However, many factors affect film formation such as; wet-ability, compatibility, rheology, pigment content, substrate properties and solvent system properties among others. The control of these factors will allow the formulator to create films with good properties such as: slipperiness, resistance to mild abrasion, leveling, good spread-ability, water resistance, adhesion and gloss.

A good film often depends on three key formulating factors, compatibility, viscosity, and wetting.

Compatibility: where various components exist in one formulation without any interactions. The components of the system should form homogeneous (one-phase) mixtures at given ratios (thermodynamic compatibility). The system should not present separation of any ingredient (two or more phases), precipitation or the presence of any suspend agents (graininess, lumps, etc.) The system can be clear or transparent (soluble) or hazy or opaque, but all ingredients should coexist in one phase. In the case of coatings the system should form a homogeneous film on the substrate. If the ingredients used in the composition are only partially compatible (that is have miscibility limits). A compatibilizer is needed to maintain the homogeneous state of the composition.

Viscosity: The viscosity of a coating also influences wetting and surface defect formation, the rate of wetting is dependent on viscosity as well as surface tension. Even if other conditions are favorable (surface tension, contact angle, etc.) Spontaneous spreading or de-wetting may not occur if viscosity is too high. Surface defects may or may not form, depending on viscosity.

Wetting: Wetting involves the interaction of a liquid with a solid; it can be the spreading of a liquid over a surface, the penetration of a liquid into a porous medium, or the displacement of one liquid by another.

Thus, there is a need for compositions having improved film properties that can be used as a base in cosmetic compositions. In particular, there is a need for base compositions for use in cosmetics that are compatible with common ingredients used in color cosmetics, allow for adjustment of the viscosity of the cosmetic films upon application, and provide adequate wetting of the surface of skin upon application. Furthermore, such base compositions should possess aesthetics qualities to allow for the formulation of appealing consumer cosmetic products.

The present inventors have discovered such a base composition containing a hydrocarbyl functional organopolysiloxane and an organopolysiloxane resin.

### SUMMARY OF THE INVENTION

The present invention provides a composition comprising;
(i) a hydrocarbyl functional organopolysiloxane comprising a siloxy unit of the formula R¹RᵢSiO_{(3-i)/2} wherein
   R is a monovalent hydrocarbon group,
   R¹ is a hydrocarbyl group having the formula —R²OCH₂CH₂OH,
   R² is a divalent hydrocarbon group containing 2 to 6 carbon atoms,
   i is zero to 2; and
(ii) an organopolysiloxane resin.

This invention also relates to cosmetic, household, or health care formulations comprising the composition of the present invention.

### DETAILED DESCRIPTION OF THE INVENTION

The hydrocarbyl functional organopolysiloxane of the present invention comprises a siloxy unit of the formula R¹RᵢSiO_{(3-i)/2} wherein R is any monovalent hydrocarbon group, but typically is an alkyl, cycloalkyl, alkenyl, aralkyl, or an aryl group containing 1-20 carbon atoms, R¹ is a hydrocarbyl group having the formula —R²OCH₂CH₂OH, R² is a divalent hydrocarbon group containing 2 to 6 carbon atoms, i is zero to 2.

Organopolysiloxanes are well known in the art and are often designated as comprising any number of M units (R₃SiO_{0.5}), D units (R₂SiO), T units (RSiO_{1.5}), or Q units (SiO₂) where R is independently any monovalent hydrocarbon group. In the present invention, the organopolysiloxane has at least one hydrocarbyl substituent of the formula —R²OCH₂CH₂OH, designated as R¹. The R² group in the hydrocarbyl substituent is a divalent hydrocarbon group containing 2 to 6 carbon atoms. The R² divalent hydrocarbon is represented by an ethylene, propylene, butylene, pentylene, or hexylene. Typically, the divalent hydrocarbon is a propylene group, —CH₂CH₂CH₂—.

The hydrocarbyl substituent is bonded to the organopolysiloxane via a Si—C bond. The hydrocarbyl substituent can be present in the organopolysiloxane via linkage to any organosiloxy unit, that is it may be present on any M, D, or T siloxy unit. In other words, the hydrocarbyl functional siloxy unit can be a M unit (R¹R₂SiO_{0.5}), a D unit (R¹RSiO), a T unit (R¹SiO_{1.5}), or a mixture of any of these. The hydrocarbyl functional organopolysiloxane can also contain any number of additional M, D, T, or Q siloxy units of the general formula (R₃SiO_{0.5}), (R₂SiO), (RSiO_{1.5}), or (SiO₂), providing that the organopolysiloxane has at least one siloxy unit with the R¹ present.

The weight average molecular weight (M_{W}) or number average molecular weight (M_{N}) of the hydrocarbyl functional organopolysiloxane can vary, and is not limiting. The hydrocarbyl functional organopolysiloxane can be either liquid or solid in form, but are generally liquids.

The amount of the hydrocarbyl functional groups present in the organopolysiloxanes of the present invention can vary, but typically ranges from 1 to 40 mass percent, alternatively from 5 to 30 mass percent, or alternatively from 10 to 20 mass percent of the total mass of the organopolysiloxane.

In one embodiment, the hydrocarbyl functional organopolysiloxane has a formula selected from the group:
R₃SiO(RR¹SiO)_{y}SiR₃,
R₃SiO(R₂SiO)ₓ(RR¹SiO)_{y}SiR₃,
R¹R₂SiO(R₂SiO)ₓSiR₂R¹,
R¹R₂SiO(RR¹SiO)_{z}SiR₂R¹,
R¹R₂SiO(R₂SiO)ₓ(RR¹SiO)_{z}SiR₂R¹,
R¹R₂SiO(R₂SiO)ₓSiR₃,
R¹R₂SiO(RR¹SiO)_{z}SiR₃,
R¹R₂SiO(R₂SiO)ₓ(RR¹SiO)_{z}SiR₃, and cyclic siloxanes of the formula
—(Me₂SiO)ₘ(MeR¹SiO)ₙ—.

In these formulas, R is an alkyl, cycloalkyl, alkenyl, aralkyl, or an aryl group containing 1-20 carbon atoms; R¹ is the hydrocarbyl group as defined above, x is 1-500, y is 1-40, z is 1-40, m is 1-6, n is 1-6, and the sum of m+n is 3-12.

In the alternate embodiment, the hydrocarbyl functional organopolysiloxane is a resin having the formula;
(SiO₂)_{c}(R³SiO_{3/2})_{d}(R³₂SiO)ₑ(R³₃SiO_{1/2})_{f}{O_{1/2}SiR³₂R⁴}_{g}
where R³ is an alkyl group with 1-20 carbon atoms, a cycloalkyl group with 3-20 carbon atoms, an alkenyl group with 2-20 carbon atoms, an aralkyl group, or an aryl group; R⁴ is the same as R¹ above, i.e., one of the formulas (i) to (iv); and g is 1-15,000. In such resins, c, d, e, and f represent mole percents, such that c<100, c+d>0, and c+d+e+f is 100. Organosiloxane resins of this type typically contain about 0.01-15 weight percent of silanol.

In a preferred embodiment, the hydrocarbyl functional organopolysiloxane has the formula
R¹Me₂SiO(Me₂SiO)ₓSiMe₂R¹
where R¹ is —(CH₂)₃OCH₂CH₂OH and x is 1 to 100, alternatively 5 to 50, or alternatively 10 to 20.

The hydrocarbyl functional organopolysiloxanes of the present invention can be made by standard processes such as the hydrosilylation of organohydrogensiloxanes and olefinically substituted polyoxyalkylenes. The hydrosilylation reaction is typically performed in a low molecular weight volatile hydrocarbon solvent such as benzene, toluene, xylene, or isopropanol to aid in handling the reactants, to moderate an exothermic reaction or to promote the solubility of the reactants. Such processes are described, for example, in the '218 patent noted above, which is incorporated herein by reference.

The composition of the present invention also contains (ii) an organopolysiloxane resin comprising various siloxy units such as (i) (R₃SiO_{1/2})ₐ, (ii) (R₂SiO_{2/2})_{b}, (iii) (RSiO_{3/2})_{c}, or (iv) (SiO_{4/2})_{d} units which are commonly known in the art, and also used herein, as M, D, T, and Q units respectively. The amount of each siloxy unit present in the organopolysiloxane resin can be expressed as a mole fraction of the total number of moles of all siloxy units present in the organopolysiloxane resin. Thus, the organopolysiloxane resins useful in the present invention comprise the units:
(i) (R₃SiO_{1/2})ₐ
(ii) (R₂SiO_{2/2})_{b}
(iii) (RSiO_{3/2})_{c}, and
(iv) (SiO_{4/2})_{d},
   wherein
R is a monovalent hydrocarbon group,
a is zero to 0.8
b is zero to 0.4,
c, is zero to 0.1.0,
d is zero to 0.8,
with the provisos that the value of either c or d is greater than zero, and the value of a+b+c+d=1.

In one embodiment of the present invention, the organopolysiloxane resin is a MQ resin comprising at least 80 mole % (R₃SiO_{1/2})ₐ and (SiO_{4/2})_{d} units where R is a monovalent hydrocarbon group, a and d has a value greater than zero, and the ratio of a/d is 0.5 to 1.5.

MQ resins suitable for use as component ii), and methods for their preparation, are known in the art. For example, U.S. Pat. No. 2,814,601 to Currie et al., Nov. 26, 1957, which is hereby incorporated by reference, discloses that MQ resins can be prepared by converting a water-soluble silicate into a silicic acid monomer or silicic acid oligomer using an acid. When adequate polymerization has been achieved, the resin is end-capped with trimethylchlorosilane to yield the MQ resin. Another method for preparing MQ resins is disclosed in U.S. Pat. No. 2,857,356 to Goodwin, Oct. 21, 1958, which is hereby incorporated by reference. Goodwin discloses a method for the preparation of an MQ resin by the cohydrolysis of a mixture of an alkyl silicate and a hydrolyzable trialkylsilane organopolysiloxane with water.

The MQ resins suitable as component ii) in the present invention may contain D and T units, providing that at least 80 mole %, alternatively 90 mole % of the total siloxane units are M and Q units. The MQ resins may also contain hydroxy groups. Typically, the MQ resins have a total weight % hydroxy content of 2-10 weight %, alternatively 2-5 weight %. The MQ resins can also be further “capped” wherein residual hydroxy groups are reacted further with M groups.

The compositions according to the invention can be formed by combining the hydrocarbyl functional organopolysiloxane component and the organopolysiloxane resin component, as described above, at a weight ratio of 1:99 to 99:1. Such compositions can generally be prepared at room temperature, using simple propeller mixers, Brookfield counter-rotating mixers, or homogenizing mixers. No special equipment or processing conditions are typically required.

The organopolysiloxane resins can be used neat, or alternatively, the organopolysiloxane resin can be dissolved in a carrier. The carrier can be any organic or silicone based solvent, which are further described infra.

Representative, non-limiting, examples of a commercial products suitable for use as component ii) include; Dow Corning ® 749 Fluid, Dow Corning ® 593, Dow Corning ® 7-4405 Cosmetic Fluid, Dow Corning® 2400 Resin, (Dow Corning Corporation, Midland Mich.),

The compositions of the present invention can be further combined with a personal care ingredient, household care ingredient, or health care ingredient. Such compositions can contain; (i) 0.1-99.9 percent of the hydrocarbyl functional organopolysiloxane fluid or the hydrocarbyl functional organopolysiloxane resin; (ii) 0.1 to 99.9 percent of the sunscreen agent; optionally (iii) 0.1-40 percent of a cosmetic active, household care active, or health care active; and (iv) the balance to 100 percent being water, an organic solvent, a silicone solvent, or one or more optional ingredients, depending upon the particular type of composition being prepared, and its intended end use or application. Generally, such compositions can generally be prepared at room temperature, using simple propeller mixers, Brookfield counter-rotating mixers, or homogenizing mixers. No special equipment or processing conditions are typically required.

As used herein, the terms personal care composition, health care composition, and household care composition are intended to mean typical materials commercially available as products or raw materials in consumer markets containing active and inactive ingredients.

The hydrocarbyl functional organopolysiloxanes are useful in a number of different products, including hair care products such as hairsprays, shampoos, mousses, styling gels and lotions, cream rinses/conditioners, hair tonics, hair dyes and colorants, permanent waves and bleaches. Also included are skin care products such as cleansers, moisturizers, conditioners, lipsticks, eye makeup, foundations, fingernail polish, suntan products, antiperspirant/deodorant products and depilatories. Also included are household products such as waxes, polishes, heavy and light duty liquid cleaners, fabric softeners, ironing aids, laundry detergents, and window cleaners. Also included are automotive products, such as, waxes, polishes, heavy and light duty cleaners, window cleaners, tire cleaners and protectors, vinyl cleaners and protectors, and the like.

Some typical ingredients used in these products are surfactants, pigments, solvents, emollients, and carriers. For example, the solvents can include esters (for example, isopropyl myristate and C₁₂₋₁₅ alkyl lactate), water, silicone fluids (for example, cyclomethicone, dimethicone), ethanol, isopropanol, guerbet alcohols having 8-30 carbons, particularly 12-22 carbons (for example, isolauryl alcohol, isocetyl alcohol, isostearyl alcohol), fatty alcohols (for example, stearyl alcohol, myristyl alcohol, oleyl alcohol), and ethoxylated and propoxylated alcohols (for example, the polyethylene glycol ether of lauryl alcohol that conforms to the formula CH₃(CH₂)₁₀CH₂(OCH₂CH₂)ᵣ OH where r has an average value of 4 (Laureth-4); PPG-14 butyl ether, where the “PPG-14” portion is the polymer of propylene oxide that conforms generally to the formula H(OCH₂C(CH₃)H)ₛOH, where s has an average value of 14, or PPG-3 myristyl ether which is the polypropylene glycol ether of myristyl alcohol that conforms to the formula CH₃(CH₂)₁₂CH₂(OCH(CH₃)CH₂)ₜOH where t has an average value of 3, or a hydrocarbon fluid.

Hydrocarbon fluids are exemplified by organic hydrocarbon fluids such as halogenated hydrocarbon fluids, aliphatic hydrocarbon fluids, aromatic hydrocarbon fluids, and mixtures of aromatic and aliphatic hydrocarbon fluids. The hydrocarbon fluids usually contain about 6 to about 12 carbon atoms. Examples of suitable hydrocarbon fluids include perchloroethylene, benzene, xylene, toluene, mineral oil fractions, kerosenes, naphthas, and petroleum fractions. Particularly preferred are isoparaffinic hydrocarbon fluids exemplified by isoparaffin fluids available from Exxon Mobil Chemical Company, Houston, Tex. U.S.A., sold as Isopar® M Fluid (a C₁₃-C₁₄ Isoparaffin), Isopar® C Fluid (a C₇-C₈ Isoparaffin), Isopar® E Fluid (a C₈-C₉ Isoparaffin), Isopar® G Fluid (a C₁₀₋₁₁ Isoparaffin), Isopar® L Fluid (a C₁₁-C₁₃ Isoparaffin), Isopar® H Fluid (a C₁₁-C₁₂ Isoparaffin), and combinations thereof. Mixtures of solvents can also be used.

Another ingredient which can be used is an emollient, including compositions such as guerbet alcohols (such as isocetyl alcohol or isostearyl alcohol); esters (such as isopropyl palmitate, isopropyl isostearate, octyl stearate, hexyl laurate and isostearyl lactate); a liquid mixture of hydrocarbons which are liquids at ambient temperatures (such as petroleum distillates and light mineral oils); ethanol; volatile and non-volatile silicone oils, highly branched hydrocarbons, and non-polar carboxylic acids. The emollients can be included in the compositions of the present invention in amounts within the range of 0.01-70%, preferably 0.1-25%, by weight, of the total weight of the composition.

The carrier can include a wide variety of conditioning materials, such as hydrocarbons, silicone fluids, and cationic materials. The carrier can include surfactants, suspending agents, thickeners etc. Various additional components useful in these compositions are described in U.S. Pat. No. 4,387,090 (Jun. 7, 1983).

Topical cosmetic, and pharmaceutical compositions according to the invention can contain a carrier, but the carrier should be cosmetically and/or pharmaceutically acceptable, i.e., that it is suitable for topical application to the skin, has good aesthetic properties, is compatible with the siloxane copolymers of the present invention, and will not cause any safety or toxicity concerns. It can be formulated to include an emulsion as the carrier such as an oil-in-water emulsion, water-in-oil emulsion, water-in-oil-in-water emulsion, or oil-in-water-in-silicone oil emulsion.

Some other suitable topical carriers include anhydrous liquid solvents such as oils, alcohols, and silicones (e.g., mineral oil, ethanol, isopropanol, dimethicone, cyclomethicone, and the like); aqueous-based single phase solvents (e.g., where the viscosity of the solvent has been increased to form a solid or semi-solid by the addition of appropriate gums, resins, waxes, polymers, salts, and the like). However, the preferred cosmetically and/or pharmaceutically acceptable topical carrier is a hydroalcoholic system or an oil-in-water emulsion. When the carrier is an oil-in-water emulsion, it will include common ingredients generally used for preparing emulsions.

Some of the typical active ingredients used in products such as these are antiacne agents, anticaries agents, antidandruff agents, antifungal agents, antimicrobial agents, antioxidants, antiperspirant agents and deodorant agents, cosmetic biocides, external analgesics, oral care agents, oral care drugs, oxidizing agents, reducing agents, skin bleaching agents, skin protectants, sunscreen agents, UV light absorbing agents, enzymes, optical brighteners, fabric softening agents, and surfactants

Some examples of antiacne agents are Salicylic acid and Sulfur. Some examples of anticaries agents are Sodium Fluoride, Sodium Monofluorophosphate, and Stannous Fluoride. Some examples of antidandruff agents are Coal tar, Salicylic acid, Selenium Sulfide, Sulfur, and Zinc Pyrithione. Some examples of antifungal agents are Calcium Undecylenate, Undecylenic Acid, Zinc Undecylenate, and Povidone-Iodine. Some examples of antimicrobial agents are Alcohol, Benzalkonium Chloride, Benzethonium Chloride, Hydrogen Peroxide, Methylbenzethonium Chloride, Phenol, Poloxamer 188, and Povidone-Iodine.

Some examples of antioxidants are Acetyl Cysteine, Arbutin, Ascorbic Acid, Ascorbic Acid Polypeptide, Ascorbyl Dipalmitate, Ascorbyl Methylsilanol Pectinate, Ascorbyl Palmitate, Ascorbyl Stearate, BHA, p-Hydroxyanisole, BHT, t-Butyl Hydroquinone, Caffeic Acid, Camellia Sinensis Oil, Chitosan Ascorbate, Chitosan Glycolate, Chitosan Salicylate, Chlorogenic Acids, Cysteine, Cysteine HCI, Decyl Mercaptomethylimidazole, Erythorbic Acid, Diamylhydroquinone, Di-t-Butylhydroquinone, Dicetyl Thiodipropionate, Dicyclopentadiene/t-Butylcresol Copolymer, Digalloyl Trioleate, Dilauryl Thiodipropionate, Dimyristyl Thiodipropionate, Dioleyl Tocopheryl Methylsilanol, Isoquercitrin, Diosmine, Disodium Ascorbyl Sulfate, Disodium Rutinyl Disulfate, Distearyl Thiodipropionate, Ditridecyl Thiodipropionate, Dodecyl Gallate, Ethyl Ferulate, Ferulic Acid, Hydroquinone, Hydroxylamine HCI, Hydroxylamine Sulfate, Isooctyl Thioglycolate, Kojic Acid, Madecassicoside, Magnesium Ascorbate, Magnesium Ascorbyl Phosphate, Melatonin, Methoxy-PEG-7 Rutinyl Succinate, Methylene Di-t-Butylcresol, Methylsilanol Ascorbate, Nordihydroguaiaretic Acid, Octyl Gallate, Phenylthioglycolic Acid, Phloroglucinol, Potassium Ascorbyl Tocopheryl Phosphate, Thiodiglycolamide, Potassium Sulfite, Propyl Gallate, Rosmarinic Acid, Rutin, Sodium Ascorbate, Sodium Ascorbyl/Cholesteryl Phosphate, Sodium Bisulfite, Sodium Erythorbate, Sodium Metabisulfide, Sodium Sulfite, Sodium Thioglycolate, Sorbityl Furfural, Tea Tree (Melaleuca Aftemifolia) Oil, Tocopheryl Acetate, Tetrahexyldecyl Ascorbate, Tetrahydrodiferuloylmethane, Tocopheryl Linoleate/Oleate, Thiodiglycol, Tocopheryl Succinate, Thiodiglycolic Acid, Thioglycolic Acid, Thiolactic Acid, Thiosalicylic Acid, Thiotaurine, Retinol, Tocophereth-5, Tocophereth- 10, Tocophereth- 12, Tocophereth- 18, Tocophereth-50, Tocopherol, Tocophersolan, Tocopheryl Linoleate, Tocopheryl Nicotinate, Tocoquinone, o-Tolyl Biguanide, Tris(Nonylphenyl) Phosphite, Ubiquinone, and Zinc Dibutyldithiocarbamrate.

Some examples of antiperspirant agents and deodorant agents are Aluminum Chloride, Aluminum Zirconium Tetrachlorohydrex GLY, Dichloro-m-Xylenol, Aluminum Chlorohydrate, Aluminum Zirconium Tetrachlorohydrex PEG, Aluminum Chlorohydrex, Aluminum Zirconium Tetrachlorohydrex PG, Aluminum Chlorohydrex PEG, Aluminum Zirconium Trichlorohydrate, Domiphen Bromide, Aluminum Chlorohydrex PG, Aluminum Zirconium Trichlorohydrex GLY, Hexachlorophene, Aluminum Dichlorohydrate, Ammonium Phenolsulfonate, Ketoglutaric Acid, Aluminum Dichlorohydrex PEG, Benzalkonium Bromide, Lauryl Isoquinolinium Bromide, Aluminum Dichlorohydrex PG, Benzalkonium Cetyl Phosphate, Laurylpyridinium Chloride, Aluminum Lactate, Benzalkonium Chloride, Methylbenzethonium Chloride, Aluminum Phenolsulfonate, Benzalkonium Saccharinate, Phenol, Aluminum Sesquichlorohydrate, Benzethonium Chloride, Sodium Bicarbonate, Aluminum Sesquichlorohydrex PEG, Bromochlorophene, Sodium Phenolsulfonate, Aluminum Sesquichlorohydrex PG, Cetylpyridinium Chloride, Tricloban, Aluminum Sulfate, Chlorophyllin-Copper Complex, Triclosan, Aluminum Zirconium Octachlorohydrate, Chlorothymol, Zeolite, Aluminum Zirconium Octachlorohydrex GLY, Zinc Lactate, Aluminum Zirconium Pentachlorohydrate, Cloflucarban, Zinc Phenolsulfonate, Aluminum Zirconium Pentachlorohydrex GLY, Dequalinium Chloride, Zinc Ricinoleate, Aluminum Zirconium Tetrachlorohydrate, and Dichlorophene.

Some examples of cosmetic biocides are Aluminum Phenolsulfonate, Ammonium Phenolsulfonate, Bakuchiol, Benzalkonium Bromide, Benzalkonium Cetyl Phosphate, Benzalkonium Chloride, Benzalkonium Saccharinate, Benzethonium Chloride, Potassium Phenoxide, Benzoxiquine, Benzoxonium Chloride, Bispyrithione, Boric Acid, Bromochlorophene, Camphor Benzalkonium Methosulfate, Captan, Cetalkonium Chloride, Cetearalkonium Bromide, Cetethyldimonium Bromide, Cetrimonium Bromide, Cetrimonium Chloride, Cetrimonium Methosulfate, Cetrimonium Saccharinate, Cetrimonium Tosylate, Cetylpyridinium Chloride, Chloramine T, Chlorhexidine, Chlorhexidine Diacetate, Chlorhexidine Digluconate, Chlorhexidine Dihydrochloride, p-Chloro-m-Cresol, Chlorophene, p-Chlorophenol, Chlorothymol, Chloroxylenol, Chlorphenesin, Ciclopirox Olamine, Climbazole, Cloflucarban, Clotrimazole, Coal Tar, Colloidal Sulfur, o-Cymen-5-ol, Dequalinium Acetate, Dequalinium Chloride, Dibromopropamidine Diisethionate, Dichlorobenzyl Alcohol, Dichlorophene, Dichlorophenyl Imidazoldioxolan, Dichloro-m-Xylenol, Diiodomethyltolylsulfone, Dimethylol Ethylene Thiourea, Diphenylmethyl Piperazinylbenzimidazole, Domiphen Bromide, 7-Ethylbicyclooxazolidine, Fluorosalan, Formaldehyde, Glutaral, Hexachlorophene, Hexamidine, Hexamidine Diisethionate, Hexamidine Diparaben, Hexamidine Paraben, Hexetidine, Hydrogen Peroxide, Hydroxymethyl Dioxoazabicyclooctane, Ichthammol, Isopropyl Cresol, Lapyrium Chloride, Lauralkonium Bromide, Lauralkonium Chloride, Laurtrimonium Bromide, Laurtrimonium Chloride, Laurtrimonium Trichlorophenoxide, Lauryl Isoquinolinium Bromide, Lauryl Isoquinolinium Saccharinate, Laurylpyridinium Chloride, Mercuric Oxide, Methenamine, Methenammonium Chloride, Methylbenzethonium Chloride, Myristalkonium Chloride, Myristalkonium Saccharinate, Myrtrimonium Bromide, Nonoxynol-9 Iodine, Nonoxynol- 12 Iodine, Olealkonium Chloride, Oxyquinoline, Oxyquinoline Benzoate, Oxyquinoline Sulfate, PEG-2 Coco-Benzonium Chloride, PEG-10 Coco-Benzonium Chloride, PEG-6 Undecylenate, PEG-8 Undecylenate, Phenol, o-Phenylphenol, Phenyl Salicylate, Piroctone Olamine, Sulfosuccinylundecylenate, Potassium o-Phenylphenate, Potassium Salicylate, Potassium Troclosene, Propionic Acid, PVP-Iodine, Quaternium-8, Quaternium-14, Quaternium-24, Sodium Phenolsulfonate, Sodium Phenoxide, Sodium o-Phenylphenate, Sodium Shale Oil Sulfonate, Sodium Usnate, Thiabendazole, 2,2′-Thiobis(4-Chlorophenol), Thiram, Triacetin, Triclocarban, Triclosan, Trioctyldodecyl Borate, Undecylenamidopropylamine Oxide, Undecyleneth-6, Undecylenic Acid, Zinc Acetate, Zinc Aspartate, Zinc Borate, Zinc Chloride, Zinc Citrate, Zinc Cysteinate, Zinc Dibutyldithiocarbamate, Zinc Gluconate, Zinc Glutamate, Zinc Lactate, Zinc Phenolsulfonate, Zinc Pyrithione, Zinc Sulfate, and Zinc Undecylenate.

Some examples of external analgesics are Benzyl Alcohol, Capsicum Oleoresin (Capsicum Frutescens Oleoresin), Methyl Salicylate, Camphor, Phenol, Capsaicin, Juniper Tar (Juniperus Oxycedrus Tar), Phenolate Sodium (Sodium Phenoxide), Capsicum (Capsicum Frutescens), Menthol, Resorcinol, Methyl Nicotinate, and Turpentine Oil (Turpentine).

Some examples of oral care agents are Aluminum Fluoride, Dicalcium Phosphate Dihydrate, Sodium Bicarbonate, Ammonium Fluoride, Domiphen Bromide, Sodium Chloride, Ammonium Fluorosilicate, Ferric Glycerophosphate, Sodium Fluoride, Ammonium Monofluorophosphate, Glycerin, Sodium Fluorosilicate, Ammonium Phosphate, Hexetidine, Sodium Glycerophosphate, Calcium Carbonate, Hydrated Silica, Sodium Metaphosphate, Calcium Fluoride, Hydrogenated Starch Hydrolysate, Sodium Monofluorophosphate, Calcium Glycerophosphate, Hydrogen Peroxide, Sodium Phytate, Calcium Monofluorophosphate, Hydroxyapatite, Sodium Styrene/Acrylates/Divinylbenzene, Calcium Phosphate, Magnesium Fluoride, Calcium Pyrophosphate, Magnesium Fluorosilicate, Stannous Fluoride, Cetylamine Hydrofluoride, Magnesium Glycerophosphate, Stannous Pyrophosphate, Cetylpyridinium Chloride, Manganese Glycerophosphate, Strontium Acetate, Chlorohexidine, Olaflur, Strontium Chloride, Chlorohexidine Diacetate, Phytic Acid, Tetrapotassium Pyrophosphate, Chlorohexidine Digluconate, Polyethylene, Tetrasodium Pyrophosphate, Chlorohexidine Dihydrochloride, Potassium Fluoride, Tricalcium Phosphate, Chlorothymol, Potassium Fluorosilicate, Zinc Chloride, Dequalinium Chloride, Potassium Glycerophosphate, Zinc Citrate, Diammonium Phosphate, Potassium Monofluorophosphate, Zinc Sulfate, and Dicalcium Phosphate.

Some examples of oral care drugs are Ammonium Alum, Potassium Alum, Benzyl Alcohol, Carbamide Peroxide, Elm Bark Extract, Gelatin, Glycerin, Hydrogen Peroxide, Menthol, Pectin, Phenol, Sodium Bicarbonate, Sodium Perborate, and Zinc Chloride.

Some examples of oxidizing agents are Ammonium Persulfate, Calcium Peroxide, Hydrogen Peroxide, Magnesium Peroxide, Melamine Peroxide, Potassium Bromate, Potassium Caroate, Potassium Chlorate, Potassium Persulfate, Sodium Bromate, Sodium Carbonate Peroxide, Sodium Chlorate, Sodium Iodate, Sodium Perborate, Sodium Persulfate, Strontium Dioxide, Strontium Peroxide, Urea Peroxide, and Zinc Peroxide.

Some examples of reducing agents are Ammonium Bisufite, Ammonium Sulfite, Ammonium Thioglycolate, Ammonium Thiolactate, Cystemaine HCl, Cystein, Cysteine HCl, Ethanolamine Thioglycolate, Glutathione, Glyceryl Thioglycolate, Glyceryl Thioproprionate, Hydroquinone, p-Hydroxyanisole, Isooctyl Thioglycolate, Magnesium Thioglycolate, Mercaptopropionic Acid, Potassium Metabisulfite, Potassium Sulfite, Potassium Thioglycolate, Sodium Bisulfite, Sodium Hydrosulfite, Sodium Hydroxymethane Sulfonate, Sodium Metabisulfite, Sodium Sulfite, Sodium Thioglycolate, Strontium Thioglycolate, Superoxide Dismutase, Thioglycerin, Thioglycolic Acid, Thiolactic Acid, Thiosalicylic Acid, and Zinc Formaldehyde Sulfoxylate.

An example of a skin bleaching agent is Hydroquinone.

Some examples of skin protectants are Allantoin, Aluminum Acetate, Aluminum Hydroxide, Aluminum Sulfate, Calamine, Cocoa Butter, Cod Liver Oil, Colloidal Oatmeal, Dimethicone, Glycerin, Kaolin, Lanolin, Mineral Oil, Petrolatum, Shark Liver Oil, Sodium Bicarbonate, Talc, Witch Hazel, Zinc Acetate, Zinc Carbonate, and Zinc Oxide.

Compositions prepared according to the invention can be used in various over-the-counter (OTC) personal care compositions, health care compositions, and household care compositions, but especially in the personal care arena. Thus, they can be used in antiperspirants, deodorants, skin creams, skin care lotions, moisturizers, facial treatments such as acne or wrinkle removers, personal and facial cleansers, bath oils, perfumes, colognes, sachets, sunscreens, pre-shave and after-shave lotions, liquid soaps, shaving soaps, shaving lathers, hair shampoos, hair conditioners, hair sprays, mousses, permanents, depilatories, hair cuticle coats, make-ups, color cosmetics, foundations, blushes, lipsticks, lip balms, eyeliners, mascaras, oil removers, color cosmetic removers, nail polishes, and powders.

### EXAMPLES

The following examples are presented to further illustrate the compositions and methods of this invention, but are not to be construed as limiting the invention. All parts and percentages in the examples are on a weight basis and all measurements were obtained at about 23° C., unless indicated to the contrary.

In the representative examples that follow, the ingredient listed as “Carbinol fluid” is Dow Corning® 5562 Carbinol fluid (Dow Corning Corporation, Midland Mich.), a hydrocarbyl functional organopolysiloxane having the formula,

R¹Me₂SiO(Me₂SiO)ₓSiMe₂R¹ where R is —(CH₂)₃OCH₂CH₂OH, and x is such to provide the product with a viscosity of about 50 cS (mm²/s) at 23° C.

### Example 1

The Carbinol Fluid was evaluated as a wetting agent in an un-pigmented coating composition containing a silicone resin and carrier. For comparison, other commonly used wetting agents were also evaluated. These included; Dow Corning ® 5211 (Dimethicone Copolyol) from Dow Corning Corporation, Midland Mich., a silicone polyether wetting agent, Crodamol PTIS (Pentaerythrityl Tetraisostearate) from Croda Inc., Edison, N.J., (US division of Croda International Plc), and castor oil. The coating compositions contained 10 wt % wetting agent, a Silicone Resin at 20 wt % solids, and 70 wt % carrier. The carriers used were Cyclomethicone (D5) and Isododecane. The silicone resins used in the coating compositions were; Dow Corning® SR 2400, Dow Corning® 749 Fluid, Dow Corning® 7-4405 Cosmetic Fluid, Dow Corning Corporation, Midland, Mich. The coatings were applied on a Lenetta chart using a Maylard coating rod No 8. The resulting films were characterized by visual observations and measurements of 60° gloss with a Gardner Tri-Gloss Meter. Uniform films were described as “pass”. The formulations and results are summarized in Table 1.

TABLE 1

| Name of wetting | | | Castor | | | 5211 | Carbinol |
|---|---|---|---|---|---|---|---|
| agent | Castor Oil | Castor Oil | Oil | 5211 Fluid | 5211 Fluid | Fluid | Fluid |
| Test material | SR 2400 | 7-4405 | 749 | SR 2400 | 7-4405 | 749 | SR 2400 |
| Name | Resin | | Fluid | Resin | | Fluid | Resin |
| Test material | Isododecane | Isododecane | D5 | Isododecane | Isododecane | D5 | Isododecane |
| base carrier | | | | | | | |
| Name of carrier | D5 | Isododecane | D5 | D5 | D5 | D5 | D5 |
| used to dilute | | | | | | | |
| test material | | | | | | | |
| Compatibility of | Fail | Fail | Fail | Pass | Pass | Fail | Pass |
| the System >24 | | | | | | | |
| hour result | | | | | | | |
| Firlm | NA | NA | NA | Pass | Fail | NA | Pass |
| Characterization | | | | | | | |
| Lenetta | | | | | | | |
| Gloss | NA | NA | NA | 68.3 | NA | NA | 62.7 |

| Name of wetting | Carbinol | Carbinol | | | |
|---|---|---|---|---|---|
| agent | Fluid | Fluid | PTIS | PTIS | PTIS |
| Test material | 7-4405 | 749 Fluid | SR 2400 | 7-4405 | 749 |
| Name | | | Resin | | Fluid |
| Test material | Isododecane | D5 | Isododecane | Isododecane | D5 |
| base carrier | | | | | |
| Name of carrier | D5 | D5 | D5 | Isododecane | D5 |
| used to dilute | | | | | |
| test material | | | | | |
| Compatibility of | Pass | Pass | Pass | Pass | Pass |
| the System >24 | | | | | |
| hour result | | | | | |
| Firlm | Pass | Pass | Pass | Fail | pass |
| Characterization | | | | | |
| Lenetta | | | | | |
| Gloss | 75.6 | 25.4 | 60.7 | NA | 19.2 |

The results show better compatibility in the system when the Carbinol Fluid was used as the wetting agent. The films based on the Carbinol fluid were uniform and had the highest gloss values.

Other variations may be made in compounds, compositions, and methods described herein without departing from the essential features of the invention. The embodiments of the invention specifically illustrated herein are exemplary only and not intended as limitations on their scope except as defined in the appended claims.

1. A composition comprising;
(i) a hydrocarbyl functional organopolysiloxane comprising a siloxy unit of the formula R¹RᵢSiO_{(3-i)/2} wherein
R¹ is a monovalent hydrocarbon group,
R² is a hydrocarbyl group having the formula —R²OCH₂CH₂OH,
R² is a divalent hydrocarbon group containing 2 to 6 carbon atoms,
i is zero to 2; and
(ii) an organopolysiloxane resin.

2. The composition of claim 1 wherein the hydrocarbyl functional organopolysiloxane contains 10 to 20 mass percent of the R¹ hydrocarbyl group.

3. The composition of claim 1 wherein the hydrocarbyl functional organopolysiloxane is a polydiorganosiloxane having a formula selected from the group:
R₃SiO(RR¹SiO)_{y}SiR₃,
R₃SiO(R₂SiO)ₓ(RR¹SiO)_{y}SiR₃,
R¹R₂SiO(R₂SiO)ₓSiR₂R¹,
R¹R₂SiO(RR¹SiO)_{z}SiR₂R¹,
R¹R₂SiO(R₂SiO)ₓ(RR¹SiO)_{z}SiR₂R¹,
R¹R₂SiO(R₂SiO)ₓSiR₃,
R¹R₂SiO(RR¹SiO)_{z}SiR₃,
R¹R₂SiO(R₂SiO)ₓ(RR¹SiO)_{z}SiR₃, and cyclic siloxanes of the formula —(Me₂SiO)ₘ(MeR¹SiO)ₙ—
where
R is an alkyl, cycloalkyl, alkenyl, aralkyl, or an aryl group containing 1-20 carbon atoms;
R¹ is —(CH₂)₃OCH₂CH₂OH;
x is 1-500, y is 1-40, z is 1-40, m is 1-6, n is 1-6, and the sum of m+n is 3-12.

4. The composition of claim 3 wherein R is methyl.

5. The composition of claim 3 wherein the polydiorganosiloxane has the formula
R¹Me₂SiO(Me₂SiO)ₓSiMe₂R¹
where R¹ is —(CH₂)₃OCH₂CH₂OH and x is 1-100.

6. The composition of claim 5 wherein x is 5-50.

7. The composition of claim 5 wherein x is 10-20.

8. The composition of claim 1 wherein the oroganopolysiloxane resin comprises the units:
(i) (R₃SiO_{1/2})ₐ
(ii) (R₂SiO_{2/2})_{b}
(iii) (RSiO_{3/2})_{c}, and
(iv) (SiO_{4/2})_{d},
wherein
R is a monovalent hydrocarbon group,
a is zero to 0.8
b is zero to 0.4,
c, is zero to 01.0,
d is zero to 0.8,
with the provisos that the value of either c or d is greater than zero, and the value of a+b+c+d=1.

9. The composition of claim 1 wherein the organopolysiloxane resin is a MQ resin comprising at least 80 mole % (R₃SiO_{1/2})ₐ and (SiO_{4/2})_{d} units where R is a monovalent hydrocarbon group, a and d has a value greater than zero, and the ratio of a/d is 0.5 to 1.5.

10. The composition of claim 1 further comprising a carrier selected from organic or silicone solvent.

11. A composition according to claim 1, further comprising (iii) at least one cosmetic, household care, or health care active ingredient selected from the group consisting of antiacne agents, anticaries agents, antidandruff agents, antifungal agents, antimicrobial agents, antioxidants, antiperspirant agents, cosmetic biocides, deodorant agents, external analgesics, oral care agents, oral care drugs, oxidizing agents, reducing agents, skin bleaching agents, skin protectants, sunscreen agents, UV light absorbing agents, pigments, moisturizers, vitamins, enzymes, optical brighteners, fabric softening agents, and surfactants.

12. A cosmetic product comprising the composition of claim 1.

13. The cosmetic product of claim 12 where the cosmetic product is a lipstick.

14. A method of treating hair or skin comprising applying to hair or skin the composition of claim 1.
